(19) United States
(12) Patent Application Publication
(10) Pub. No.: US 2025/0359789 A1
(43) Pub. Date: Nov. 27, 2025

(54) BIOMOLECULE DETECTION DEVICE WITH STRETCHABLE MATERIAL

(71) Applicants: Research & Business Foundation Sungkyunkwan University, Suwon-si (KR); Ninebiowear Co., Ltd., Suwon-si (KR)

(72) Inventors: Nae Eung LEE, Seoul (KR); Vinayagam Chinnamani MOTTOUR, Suwon-si (KR)

(21) Appl. No.: 19/202,066

(22) Filed: May 8, 2025

(30) Foreign Application Priority Data
May 22, 2024 (KR) 10-2024-0066380

(51) Int. Cl. A61B 5/1473; A61B 5/15

(52) U.S. Cl. CPC A61B 5/1473; A61B 5/150022; A61B 5/150099; A61B 5/150221; A61B 5/150229; A61B 5/150251; A61B 5/150389; A61B 5/150755; A61B 2560/0468

(57) ABSTRACT

Provided is a biomolecule detection device including a substrate made of a stretchable material so as to be attachable to a body. The biomolecule detection device includes a pattern of a concave valley, which is formed on the substrate of the stretchable material, wherein the pattern includes: a pressing region in which liquid inside a first pouch part is transferred to a flow path connected to the first pouch part by a user's operation; a moving region having a plurality of flow paths so that the liquid moves; a blood collection region in which blood is inhaled; and a sensing region in which biomolecules included in the blood are sensed.

### TECHNICAL FIELD

The present disclosure relates to a biomolecule detection device with a stretchable material, and more particularly, to a biomolecule detection device in which blood extraction, plasma separation, and detection of protein biomolecules in separated plasma are continuously performed in a substrate of a stretchable material.

### BACKGROUND ART

Recently, medical diagnostic technology based on microfluidic analysis technology has been developed. In this regard, blood includes the most common biomolecules that provide important information for quick and accurate diagnostics. Accordingly, development of a microfluidic detection device for blood analysis has been actively conducted.

However, the existing biomolecule detection technology includes various processes such as blood collection (blood sampling), plasma separation (pre-treatment), introduction of pretreated blood into a device, etc. This subdivided process requires a skilled worker, and requires a long time, resulting in discomfort to the clinician and patients. In addition, since the blood collection and the plasma separation are divided into different processes, respectively, there is a risk of contamination or damage to the sample, which makes it difficult to provide a rapid and accurate detection result. A conventional microfluidic detection device has a limitation in order for a non-expert to directly operate an immunoassay device due to complex use. In addition, there is a fatal disadvantage in that a patient cannot directly use the conventional microfluidic detection device in an emergency situation, such as an acute cardiovascular disease. Accordingly, there is a need for a microfluidic detection device which is manufactured in various sizes and shapes, is convenient to carry, and is made of a stretchable material to be easily attached to a body.

In particular, recently, research on a user-friendly device, which is connected to an external device such as a smart phone to monitor a patient's emergency or health and is provided as a lab-on-a-chip to accomplish convenience of use, is continued.

### SUMMARY OF THE DISCLOSURE

### Technical Problem

A task of the present disclosure is to provide a detection device capable of collecting blood by negative pressure by generating the negative pressure on a substrate made of a stretchable material, and detecting biomolecules included in plasma by separating the plasma from the collected blood.

The technical task of the present disclosure is not limited to the above-described technical task, and other technical tasks not mentioned may be clearly understood by those skilled in the art to which the present disclosure pertains from the following description.

### Technical Solution

According to an aspect of the present disclosure, there is provided a biomolecule detection device including a substrate made of a stretchable material so as to be attachable to a body, the biomolecule detection device including a pattern of a concave valley, which is formed on the substrate of the stretchable material, wherein the pattern includes: a pressing region in which liquid inside a first pouch part is transferred to a flow path connected to the first pouch part by a user's operation; a moving region having a plurality of flow paths so that the liquid moves; a blood collection region in which blood is inhaled; and a sensing region in which biomolecules included in the blood are sensed.

In the present disclosure, the pressing region may include a second pouch part which is filled with hydrophilic powder, and forms a negative pressure by performing a gelation reaction in combination of the hydrophilic powder with the liquid, and a first valve part connecting the first pouch part with the second pouch part.

In the present disclosure, the pressing region may transmit the negative pressure formed by the gelation reaction to a flow path connected to the first pouch part.

In the present disclosure, the moving region may include a first valve part connected to the first pouch part, an impeller part connected to the first valve part and including a plurality of impellers, and a second valve part connecting the impeller part with the sensing region.

In the present disclosure, the impeller part may include connection parts provided between the plurality of impellers and connecting different impellers with each other.

In the present disclosure, each of the impellers may include an impeller body connected to the corresponding connection part and through which the liquid passes through a circular space, an impeller center part, and a plurality of impeller blades arranged in a circumferential direction from the impeller center part.

In the present disclosure, the liquid may be introduced into the impeller body through the corresponding connection part, and the backflow of the liquid may be prevented while rotating in one direction by the impeller blades.

In the present disclosure, the blood collection region may include a needle part including a hollow needle to collect blood, a blood collection storage part in which the collected blood is stored, a separation part including a filter to separate plasma from the blood, a lamination part stacking blood from which the plasma is separated, and a channel part provided on one side of the separation part and configured in a zigzag shape to carry the plasma.

In the present disclosure, the blood collection region may move the blood in the direction of the negative pressure.

In the present disclosure, the sensing region may include a sensing part having a concave groove so that the plasma is filled, and a sensor unit in which a plurality of electrodes are deposited on the sensor unit to sense biomolecules of the plasma.

In the present disclosure, the negative pressure may move the blood inhaled in the blood collection region to the sensing region with a force in which the liquid positioned in the moving region flows backward in a direction toward the first pouch part.

In the present disclosure, the substrate of the stretchable material may include an upper layer and a lower layer bonded to each other by a solvent bond.

### BRIEF DESCRIPTION OF THE DRAWINGS

FIG. 1 is an exploded perspective view of a stretchable biomolecule detection device according to an embodiment of the present disclosure.

FIG. 2 is a schematic diagram of a stretchable biomolecule detection device according to an embodiment of the present disclosure.

FIG. 3 is a diagram illustrating an operation of a pressing region provided in a stretchable biomolecule detection device according to an embodiment of the present disclosure.

FIG. 4 is a schematic diagram of a moving region provided in a stretchable biomolecule detection device according to an embodiment of the present disclosure.

FIG. 5 is a detailed view of an impeller provided in a moving region of a stretchable biomolecule detection device according to an embodiment of the present disclosure.

FIG. 6 is a detailed view of a blood collection region provided in a stretchable biomolecule detection device according to an embodiment of the present disclosure.

FIG. 7 is a diagram illustrating a blood collection process of a blood collection region provided in a stretchable biomolecule detection device according to an embodiment of the present disclosure.

FIG. 8 is a detailed view of a channel part provided in a blood collection region of a stretchable biomolecule detection device according to an embodiment of the present disclosure.

FIG. 9 are detailed views of a sensing region provided in a stretchable biomolecule detection device according to an embodiment of the present disclosure.

FIG. 10 is a detailed view of a sensor unit provided in a sensing region of a stretchable biomolecule detection device according to an embodiment of the present disclosure.

### DETAILED DESCRIPTION OF THE DISCLOSURE

Hereinafter, the present disclosure will be described with reference to the accompanying drawings. However, the present disclosure may be implemented in various different forms, and thus is not limited to the embodiments described herein. In the drawings, parts irrelevant to the description have been omitted in order to clearly describe the present disclosure, and similar parts are denoted by similar reference numerals throughout the disclosure.

Throughout the disclosure, when a part is referred to as being “connected (coupled, contacted, or combined)” to another part, it includes not only “directly connected” but also “indirectly connected” with another member therebetween. In addition, when a part “includes” a certain component, this means that other components may be further included, rather than excluding other components unless otherwise specified.

The terminology used herein is for the purpose of describing particular embodiments only and is not intended to be limiting of the present disclosure. Singular expressions include plural expressions unless the context clearly indicates otherwise. It will be understood that the terms “includes”, “comprises”, “including”, and/or “comprising” when used in this disclosure, specify the presence of stated features, integers, steps, operations, elements, components, and/or combinations thereof, but do not preclude the presence or addition of one or more other features, integers, steps, operations, elements, components, and/or combinations thereof.

The present disclosure relates to a biomolecule detection device including a stretchable material to be attachable to a body, and more particularly, to a detection device in which a sensor is provided to sense biomolecules included in plasma by continuously performing extraction of blood, plasma separation, and detection of protein biomolecules in the separated plasma in a stretchable substrate by negative pressure inside the stretchable substrate.

Hereinafter, embodiments of the present disclosure will be described in detail with reference to the accompanying drawings.

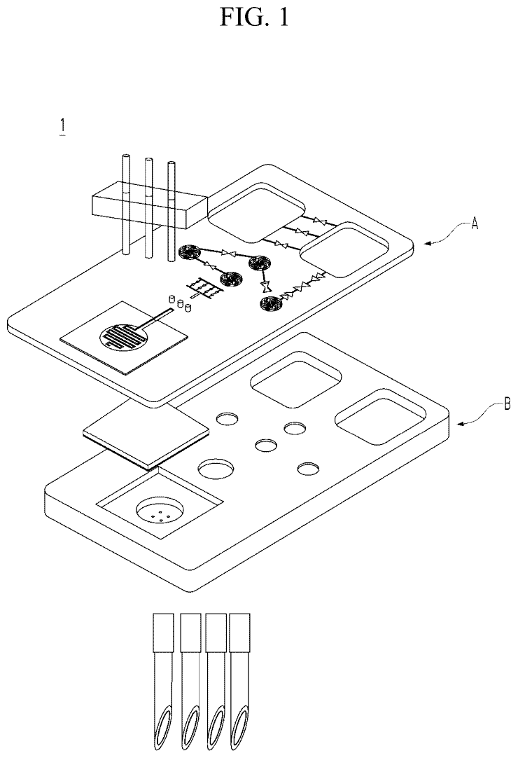

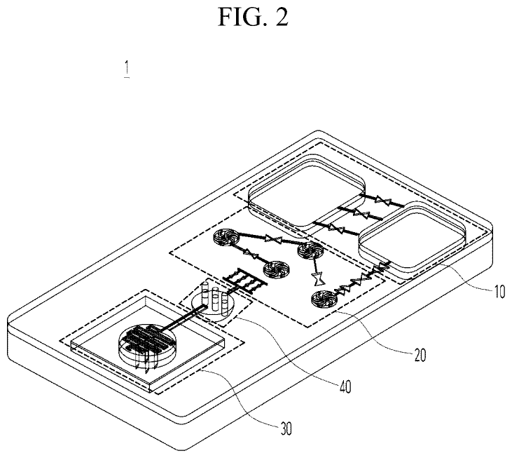

FIG. 1 is an exploded perspective view of a stretchable biomolecule detection device according to an embodiment of the present disclosure, and FIG. 2 is a schematic diagram of a stretchable biomolecule detection device according to an embodiment of the present disclosure.

Referring to FIG. 1, a detection device 1 of the present disclosure may be provided on a substrate made of a stretchable material.

The substrate of the stretchable material may include an upper layer A and a lower layer B, and the upper layer A and the lower layer B may be bonded to each other by a solvent bond.

The upper layer A and the lower layer B may include a styrene-ethylene-butylene-styrene (SEBS) base, which is a thermoplastic elastomer (TPE) material. The SEBS has excellent flexibility and stretchability as an elastic polymer-based material, and thus may be used as a substrate material of the detection device 1 of the present disclosure. In addition, the surfaces of the upper layer A and the lower layer B may be coated with (3-aminopropyl) triethoxysilane (APTES) so that the upper layer A and the lower layer B are bonded to each other by solvent bond.

Meanwhile, a plurality of flow paths through which liquid may pass may be provided between the upper layer A and the lower layer B, and the plurality of flow paths may be formed by injection molding.

Referring to FIG. 2, the detection device 1 may include a pattern of a pressing region 10, a moving region 20, a blood collection region 30, and a sensing region 40. In the detection device 1 of the present disclosure, a pattern formed of a concave valley may be formed between the upper layer A and the lower layer B, and the pattern may form the pressing region 10, the moving region 20, the blood collection region 30, and the sensing region 40.

The detection device 1 of the present disclosure may be connected in the order of the pressing region 10, the moving region 20, the sensing region 40, and the blood collection region 30, and may be operated by an external pressure applied to the pressing region 10. For example, the detection device 1 may be operated by a liquid L filled in the pressing region 10 and a pressure generated by the circulation of the liquid L.

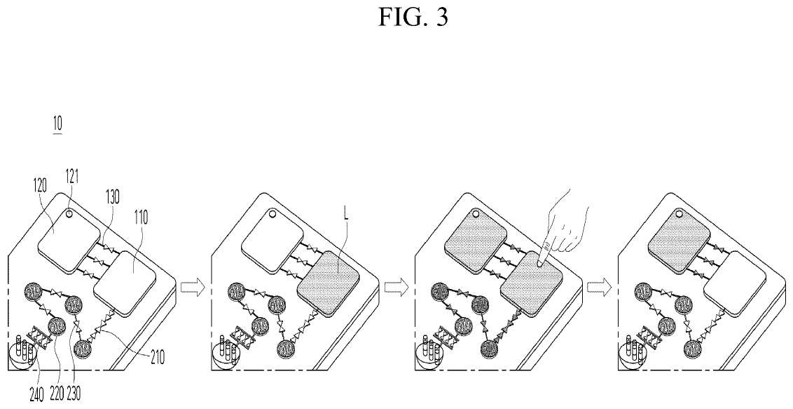

FIG. 3 is a diagram illustrating an operation of a pressing region provided in a stretchable biomolecule detection device according to an embodiment of the present disclosure.

Referring to FIG. 3, the pressing region 10 may include a first pouch part 110, a second pouch part 120, and a maintenance valve part 130.

The pressing region 10 may transmit the liquid of the first pouch part 110 to the flow path connected to the first pouch part 110 by a user's operation.

The first pouch part 110 has a concave pattern in each of the upper layer A and the lower layer B, and the first pouch part 110 may be filled with a predetermined amount of liquid L. A cover having elasticity may be provided on one side of the upper layer A. The elastic cover of the first pouch part 110 may maintain the first pouch part 110 in a sealed state so that the liquid L does not leak to the outside of the detection device 1 even when the user presses the first pouch part 110.

In addition, the liquid L in the first pouch part 110 may be transferred through a flow path connected to the first pouch part 110 by the pressing of the first pouch part 110, so that the liquid L may be distributed in the detection device 1.

The second pouch part 120 may be filled with hydrophilic powder, and the hydrophilic powder may be combined with the liquid L to be gelated through a gelation reaction.

Like the first pouch part 110, the second pouch part 120 may be made of an elastic material, and the liquid L of the first pouch part 110 may be introduced into the second pouch part 120, and the second pouch part 120 may also be filled with liquid L. In this case, the introduced liquid L may be an amount sufficient to allow the hydrophilic powder filling the second pouch part 120 to be gelated, and the liquid L remaining after being used in the gelation reaction may be discharged through an exhaust port 121 provided at one side of the second pouch part 120.

When the liquid L is introduced into the second pouch part 120, a negative pressure may be formed while performing a gelation reaction with the hydrophilic powder, and the negative pressure may be transferred to flow paths connected to each other in the inside of the detection device 1. The gelation reaction may preferably take 30 seconds to 40 seconds. This gelation reaction time is advantageous in that the liquid L is uniformly distributed into the detection device 1 through another flow path connected to the first pouch part 110, and the pressure is concentrated to the second pouch part 120 while the negative pressure is formed after the gelation reaction.

Meanwhile, the hydrophilic powder in the second pouch part 120 may be a super absorbent hydrophilic material such as carboxymethyl cellulose (CM C), and the liquid L filling the inside of the first pouch part 110 is combined with the hydrophilic powder to cause a gelation reaction, and the type thereof is not limited.

The maintenance valve part 130 may connect the first pouch part 110 with the second pouch part 120. The maintenance valve part 130 is formed by connecting the first pouch part 110 and the second pouch part 120 with an elongated passage, and a protrusion is formed in the center of the flow path to provide a space in which the fluid passing through the maintenance valve part 130 may temporarily stay. In other words, vertices of different triangular protrusions may be provided while facing each other in the middle of the longitudinal direction of the maintenance valve part 130, and may be provided in the form of “ [symbol] ” as illustrated. Meanwhile, as shown in FIGS. 2 and 3, the maintenance valve part 130 may include three connection flow paths between the first pouch part 110 and the second pouch part 120, but the number thereof is not particularly limited.

In addition, the maintenance valve part 130 may be provided to allow the liquid L filling the inside of the first pouch part 110 to pass through the second pouch part 120 while the user presses the first pouch part 110. In this case, a gelation reaction may occur in the second pouch part 120 due to the moved liquid L. At the same time, the liquid L of the first pouch part 110 may spread into the detection device 1 through other flow paths connected to the first pouch part 110. When the gelation reaction is completed in the second pouch part 120, the negative pressure may be generated, and the liquid L distributed in the detection device 1 may be concentrated in the direction toward the second pouch part 120. The liquid L may pass through the maintenance valve part 130 by the operation of the user, and the negative pressure may move while the gelation reaction is completed in the second pouch part 120.

As described above, the pressing region 10 may transmit the negative pressure formed by the gelation reaction to the flow path connected to the first pouch part 110, and the driving method in the pressing region 10 may generate negative pressure without an external pump to thereby extract blood. Here, the fluid may include the liquid L supplied from the first pouch part 110 and the negative pressure concentrated toward the second pouch part 120.

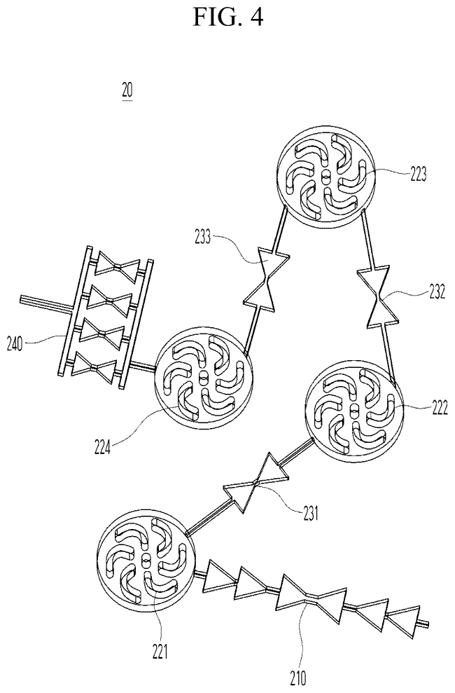

FIG. 4 is a schematic diagram of a moving region provided in a stretchable biomolecule detection device according to an embodiment of the present disclosure.

Referring to FIG. 4, a plurality of flow paths may be provided to move the liquid L, and may include a first valve part 210, an impeller part 220, a connection part 230, and a second valve part 240.

The liquid L introduced into the moving region 20 may sequentially pass through the first valve part 210, the impeller part 220, the connection part 230, and the second valve part 240, and flow back by the negative pressure. The process of back-flow by the negative pressure will be described later.

The first valve part 210 may be connected to the first pouch part 110, and the liquid L and the pressure of the first pouch part 110 may be transferred from the first valve part 210 to the moving region 20. The first valve part 210 may have a plurality of triangular protrusions in a narrow and long path, that is, an elongated path, and the protrusions provided in the first valve part 210 may be arranged symmetrically toward the center of the flow path, and the protrusions facing the center may be arranged in a “ [symbol] ” shape as illustrated while having vertices opposite to each other. The first valve part 210 may be arranged to be symmetrical from the center of the flow path longitudinal direction. In this shape, the protrusion of the first valve part 210 may allow the fluid passing through the first valve part 210 to temporarily stay therewith.

The fluid passing through the first valve part 210 may be transferred to the impeller part 220.

The impeller part 220 may be connected to the first valve part 210 and may include a plurality of impellers 221, 222, 223, and 224. The impeller part 220 may include the plurality of impellers 221, 222, 223, and 224 to allow the fluid introduced into the first valve part 210 to move in one direction. The plurality of impellers 221, 222, 223, and 224 may include a first impeller 221, a second impeller 222, a third impeller 223, and a fourth impeller 224.

The connection part 230 may include a first connection part 231, a second connection part 232, and a third connection part 233 provided between the plurality of impellers 221, 222, 223, and 224 to connect the different impellers 221, 222, 223, and 224 to each other. The plurality of impellers 221, 222, 223, and 224 may be connected to each other through the connection parts 230 to be arranged in the moving region 20, and the fluid introduced into the first valve part 210 may circulate in the moving region 20.

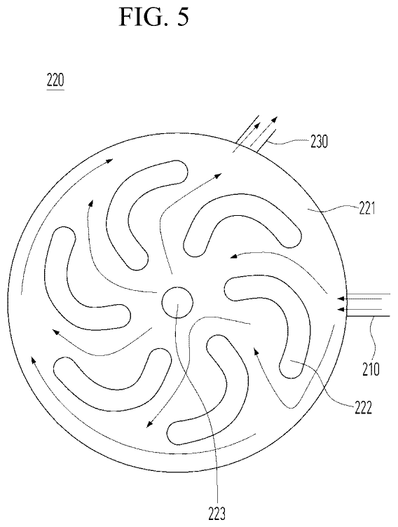

FIG. 5 is a detailed view of an impeller provided in a moving region of a stretchable biomolecule detection device according to an embodiment of the present disclosure.

Each of the plurality of impellers 221, 222, 223, and 224 may be arranged in a circular shape in the moving region 20 such that the fluid moves in one direction within the moving region 20. The plurality of impellers 221, 222, 223, and 224 may minimize the movement in the direction toward the sensing region 20 when the liquid L of the first pouch part 110 moves to the second pouch part 120 due to an external pressure applied to the first pouch part 110. The movement of the liquid L may be limited by the internal shapes of the plurality of impellers 221, 222, 223, and 224. The shapes of the plurality of impellers 221, 222, 223, and 224 will be described representatively with reference to the first impeller 221.

Referring to FIG. 5, the first impeller 221 may include an impeller body 221B, an impeller center part 221C, and an impeller blade part 221W.

The impeller body 221B may be provided in a circular shape to be connected to the at least one connection part 230 to allow liquid to pass therethrough, and the impeller blade part 221W may include a plurality of fan-shaped blades with respect to the impeller center part 221C.

The impeller blade part 221W may have the plurality of fan-shaped blades, and one side of each blade may be bent toward the impeller center part 221C, and the other side thereof may be bent in a direction of the outer circumferential surface of the impeller body 221B. The plurality of blades may be arranged in the same direction at equal intervals in the circumferential direction with respect to the impeller center part 221C.

In this case, the liquid L may flow between the impeller blade part 221W and the outer circumferential surface of the impeller body 221B, and the direction in which the liquid L flows may be formed in the direction of the outer circumferential surface of the impeller body 221B at the most protruding part of the impeller blade part 221W.

For example, the moving direction of the liquid L may flow in the outer circumferential direction of the impeller body 221B from the center of the impeller blade part 221W, and as shown in FIG. 5, the flow of the liquid L may rotate in the clockwise direction while flowing from the center of the impeller blade part 221W to the outer circumferential direction of the impeller body 221B. The liquid L introduced into the first impeller 221 through the first valve part 210 may be moved to the first connection part 231 by rotating the inside of the impeller body 221B, and may be moved to the second impeller 222.

In the second impeller 222, the third impeller 223, and the fourth impeller 224, the flow scheme of the liquid L may be the same as that of the first impeller 221, the first connection part 231 may be provided between the first impeller 221 and the second impeller 222, the second connection part 232 may be provided between the second impeller 222 and the third impeller 223, and the third connection part 233 may be provided between the third impeller 223 and the fourth impeller 224, and the liquid L may move therethrough. Meanwhile, the fourth impeller 224 may be connected to the second valve part 240.

The second valve part 240 may be provided between the impeller part 220 and the blood collection region 30 to connect each other.

The second valve part 240 may be arranged such that two long parallel flow paths are alternately arranged, and a plurality of auxiliary flow paths may be arranged at equal intervals between the two parallel flow paths, and preferably, four auxiliary flow paths may be provided. A protrusion may be formed in each auxiliary flow path provided in the second valve part 240, and vertices in a shape of a triangle may be provided facing each other while having a shape similar to a shape provided in the maintenance valve part 130.

The auxiliary flow paths of the second valve part 240 may adjust the flow of the liquid L so that the liquid L does not intrude into the sensing region 40 connected to the second valve part 240 while the liquid L passing through the second valve part 240 is temporarily stayed. When the liquid L intrudes into the sensing region 40, the liquid L is mixed with the blood B collected in the sensing region 40 to affect the plasma concentration, which may cause undesirable results.

Meanwhile, although the liquid L may flow in the direction from the first valve part 210 toward the second valve part 240 in the moving region 20, if a negative pressure is generated in the pressing region 10, the liquid L distributed in the moving region 20 may flow backward in the direction toward the pressing region 10.

For example, the liquid L distributed to the second valve part 240 may be circulated in the reverse direction from the impeller part 220 by the negative pressure generated in the pressing region 10 to flow backward to the first valve part 210.

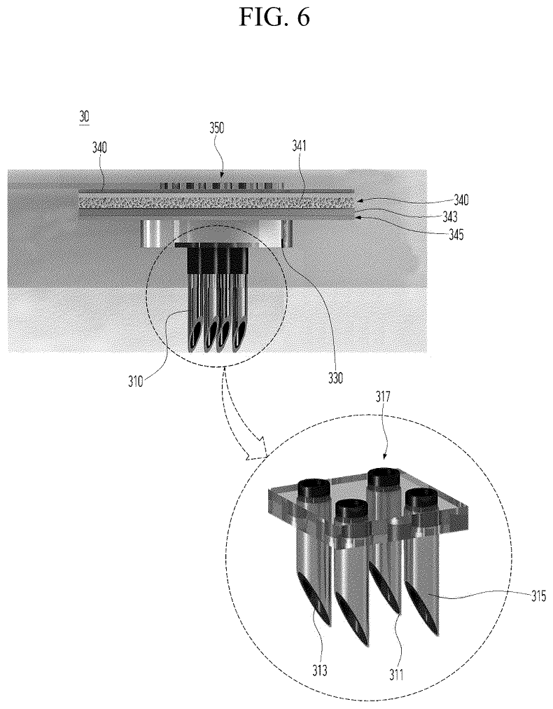

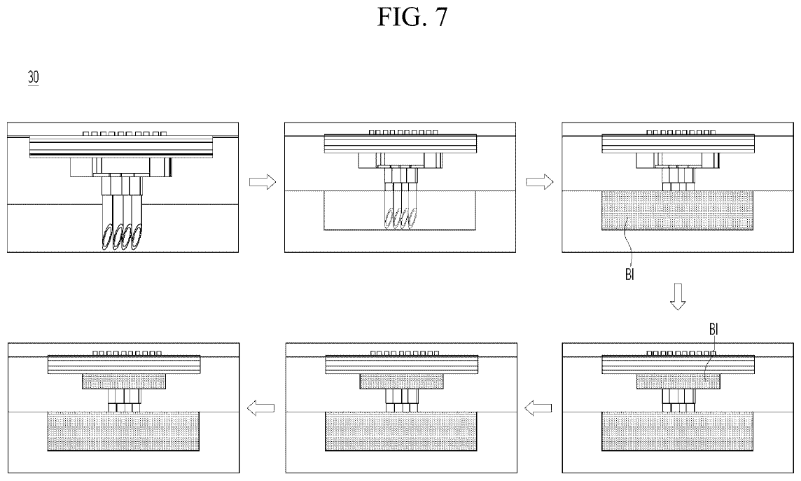

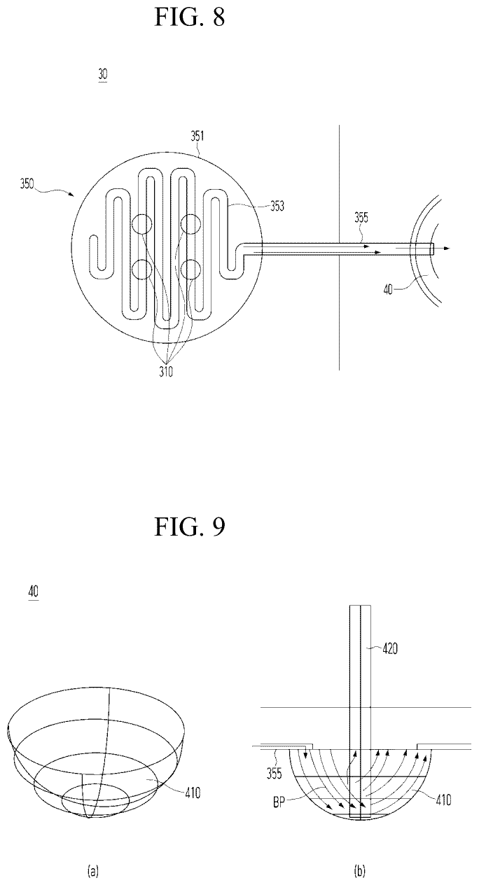

FIG. 6 is a detailed view of a blood collection region provided in a stretchable biomolecule detection device according to an embodiment of the present disclosure. FIG. 7 is a diagram illustrating a blood collection process of a blood collection region provided in a stretchable biomolecule detection device according to an embodiment of the present disclosure. FIG. 8 is a detailed view of a channel part provided in a blood collection region of a stretchable biomolecule detection device according to an embodiment of the present disclosure.

Referring to FIG. 6, the blood collection region 30 may be provided by passing through the lower layer B so as to inhale the blood BI from the skin or the region in which the blood BI is immersed. The blood collection region 30 may inhale the blood BI at a negative pressure, separate the plasma from the inhaled blood, and transfer the separated plasma to the sensing region 40. The blood collection region 30 may include a needle part 310, a blood collection storage part 320, a stacking part 330, a separation part 340, and a channel part 350.

The needle part 310 may be configured with a plurality of hollow needles so as to collect blood. The needle part 310 may include hollow microneedles made of a metal material. The plurality of hollow microneedles may be arranged at the same interval. Here, the number of the hollow microneedles of the needle part 310 may be four, but the number thereof is not particularly limited.

In addition, each of the hollow microneedles of the needle part 310 may include a tip part 311, an inclined part 313, a hollow part 315, and a needle connection part 317. The tip part 311 of each of the hollow microneedles of the needle part 310 may be formed in a sharp shape to collect blood BI by being inserted into the skin, the tip part 311 may penetrate the skin, and the blood BI may be inhaled through the hollow part 315. In this case, the inclined part 313 may be provided at one side of the tip part 311 to facilitate entry of the tip part 311. The inhaled blood BI may move to the blood collection storage part 323 provided on the upper side of the needle part 310 through the needle connection part 317.

The blood collection storage part 320 may be a space having a predetermined depth so that the collected blood is stored. The inside of the blood collection storage part 320 may be coated with an anticoagulant such as lithium heparin, which prevents the blood passing through the blood collection storage part 320 from being leached. The blood BI stored in the blood collection storage part 320 may be sealed so that the inflow and exposure of the external gas may be blocked.

The inhaled blood BI may move to the stacking part 330 located at the upper end of the blood collection storage part 320 by the negative pressure inside the detection device 1. The separation part 340 may include a filter to separate plasma from blood. The separation part 340 may include a filter part 341, an adhesive part 343, and a sheet part 345, and plasma may be separated from the filter part 341 provided in the separation part 340.

The filter part 341 may be provided as a porous membrane to separate plasma included in blood. Since the porous membrane has a large number of structures per area, plasma separation efficiency may be high, and a time required to separate plasma from blood may be reduced. In this case, the porous membrane used in the filter part 341 may include a material having compressibility after foaming, such as polyethylene, polyethersulfone, polyurethane, acrylic acid, or the like, but is not limited thereto. In addition, the filter part 341 may be a hydrophobic or hydrophilic material or a Janus filter having one hydrophobic surface and the other hydrophilic surface. Due to the negative pressure inside the detection device 1, the filter part 341 may stand against gravity while the blood BI moves from the lower part to the upper part.

The adhesive part 343 may be provided between the filter part 341 and the sheet part 345, and may include an adhesive material including an acrylic adhesive, a hot melt adhesive, a structural adhesive, or a combination thereof to bond the filter part 341 and the sheet part 345 to each other, and may be used in the form of an adhesive tape. In addition, the filter part 341 and the sheet part 345 may be attached by a hot press method, but embodiments are not limited thereto.

Meanwhile, the sheet part 345 provided on one side of the adhesive part 343 may be provided on the bottom surface of the lower layer B, and may be coated with aminopropyltriethoxysilane (APTES). The sheet part 345 may be provided to protect the filter part 341 from the blood BI leaking in the blood collection process.

Referring to FIG. 7, when the needle part 310 is deposited in the human body or a place containing the blood BI, the blood BI may be inhaled by the needle part 310 and move to the blood collection storage part 320, and plasma separated from the blood BI while passing through the stacking part 330 and the separation part 340 may be moved to the channel part 350.

The plasma separated by the separation part 340 may be moved to the channel part 350. The channel part 350 may be provided at an upper side of the separation part 340 and may be provided in a zigzag shape to transport plasma.

Referring to FIG. 8, the channel part 350 may be provided at an upper end of the needle part 310 and include a channel part body 351, a channel flow path 353, and a channel connection part 355. The channel part body 351 has a circular region, and The channel flow path 353 is provided as a zigzag flow path in the channel part body 351 to allow the plasma to move.

A plasma may be introduced into the channel part 350 from the separation part 340 provided at a lower end of the channel part 350, and the plasma may move through the channel flow path 353, pass through the channel connection part 355, and be transmitted to the detection region 40.

In this case, the blood BI in the blood collection region 30 may be moved by the negative pressure, and may move in the direction toward the sensing region 40 from the blood collection region 30.

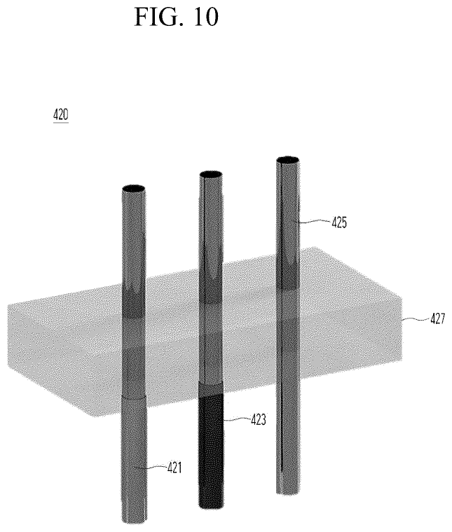

FIG. 9 are detailed views of a sensing region provided in a stretchable biomolecule detection device according to an embodiment of the present disclosure, and FIG. 10 is a detailed view of a sensor unit provided in a sensing region of a stretchable biomolecule detection device according to an embodiment of the present disclosure.

Referring to FIG. 9, the sensing region 40 may include a sensing part 410 and a sensor unit 420, in which FIG. 9 (a) is a perspective view of the sensing part 410. The sensing region 40 senses biomolecules included in plasma.

The sensing region 40 may include a hemisphere-shaped sensing part 410 filled with plasma BP, and a sensor unit 420 in which a plurality of cylinders are immersed in the sensing part 410 and sensing biomolecules of plasma. The sensing part 410 may have a concave groove shape instead of a hemisphere.

One side of the sensing part 410 may be connected to the channel connecting part 355, and the other side of the sensing part 410 may be connected to the second valve part 240. The sensing part 410 may be filled with plasma by receiving plasma through the channel connection part 355. Through the sensing part 410 connected to the second valve part 240, the plasma collected in the blood collection region 30 may be collected by the sensing part 410 by the negative pressure. That is, the blood BI inhaled by the blood collection region 30 may move to the sensing region 40 by a negative pressure, which is a force by which the liquid L located in the moving region 20 flows backward in the direction toward the first pouch part 110.

The sensor unit 420 may be provided to be in contact with the bottom surface of the sensing part 410 to sense an electrochemical signal of plasma collected by the sensing part 410 to thereby sense biomolecules of the plasma. The sensor unit 420 may be provided in the form of a surface of a metal to sense an electrochemical signal.

Referring to FIG. 10, the sensor unit 420 may include a reference electrode 421, a working electrode 423, a counter electrode 425, and an electrode pad 427. The electrodes provided in the sensor unit 420 may pass through the electrode pad 427 and may be arranged to be spaced apart from each other.

The reference electrode 421 may be provided to provide a reference potential, the working electrode 423 may be provided to measure an amount of current, and the counter electrode 425 may be provided as an electrode for applying a predetermined voltage to the reference electrode 421 and the working electrode 423. The plurality of electrodes are positioned at regular intervals on the electrode pad 427. The electrode pad 427 may include a non-conductive material such as polydimethylsiloxane (PDMS) so that the plurality of electrodes do not communicate with each other, that is, current flows between the plurality of electrodes.

The sensor unit 420 changes the amount of current flowing in the sensing part 410 while applying a voltage from the reference electrode 421 to the counter electrode 425, and quantitatively analyzes plasma accommodated in the sensing part 410 while sensing a change in the amount of current flowing from the working electrode 423 to the counter electrode 425.

The sensor unit 420 may include a material having stretchability and flexibility, such as polyimide, N-methyl-2-pyrrolidone (NM P), poly (vinylidene fluoride-co-trifluoroethylene (PVDF-TrFE), and single-walled carbon nanotubes (SWCNT). In addition, one end of the sensor unit 420 is plated with Ni and Au so as to form a microneedle, and specifically, the sensor unit 420 may be plated with a material such as Au nanocoral nanostructure (AuNS) and prussian blue (PB) to perform electrochemical sensing. The sensor unit 420 has an effect of detecting biomolecules, such as cTnI, cTnT, CK-MB, CRP, and IL-6, included in the plasma.

In addition, an external terminal can be connected to a stretchable biomolecule detection device according to the present disclosure. That is, the detection device 1 may be connected to an external terminal to immediately check the detection result of the biomolecules. The sensor unit 420 of the detection device 1 may be connected to an external terminal through an electric circuit reader, and the detection result may be checked through a display screen of the external terminal.

According to the present disclosure, the detection device 1 is light and has a small volume, so that the detection device 1 may be conveniently carried and may be easily attached to the human body by using a stretchable material, In addition, since extraction of blood, plasma separation, and detection of protein biomolecules in separated plasma are continuously performed in the detection device 1, it is easy to use the detection device 1.

Since the detection device 1 of the present disclosure moves the fluid by the pressure generated by itself without using external driving, the detection device 1 may simplify the driving method compared to the micro filter element separating plasma from blood through external driving, thereby separating plasma from blood continuously and efficiently.

The biomolecule detection device according to the present disclosure is easy to attach to a human body by using a stretchable material, is lightweight and has a small volume, and thus is convenient to carry.

In addition, since the extraction of blood, plasma separation, and the detection process of protein biomolecules in the separated plasma are continuously performed by negative pressure inside the detection device, contamination or damage of samples may be prevented.

In addition, it is possible to monitor an emergency situation or a health state of a patient by connecting the detection device according to the present disclosure to an external device such as a smartphone, and the detection device according to the present disclosure may be provided as a lab-on-a-chip to be easily used by a user.

It should be understood that the effects of the present disclosure are not limited to the above effects, and include all effects which can be inferred from the configuration of the disclosure described in the description or the claims of the present disclosure.

The foregoing description is for illustration only, and it will be understood by those of ordinary skill in the art that the present disclosure can be easily modified in other specific forms without changing the technical spirit or essential features of the present disclosure. It is therefore to be understood that the embodiments described above are illustrative in all aspects and not restrictive. For example, each component described as a single type may be distributed and implemented, and similarly, components described as being distributed may be implemented in a combined form.

The scope of the present disclosure is represented by the following claims, and all changes or modifications derived from the meaning and scope of the claims and their equivalents should be construed as being included in the scope of the present disclosure.

What is claimed is:

1. A biomolecule detection device including a substrate made of a stretchable material so as to be attachable to a body, the biomolecule detection device comprising a pattern of a concave valley, which is formed on the substrate of the stretchable material, wherein
the pattern comprises:
a pressing region in which liquid inside a first pouch part is transferred to a flow path connected to the first pouch part by a user's operation;
a moving region having a plurality of flow paths so that the liquid moves;
a blood collection region in which blood is inhaled; and
a sensing region in which biomolecules included in the blood are sensed.

2. The biomolecule detection device of claim 1, wherein the pressing region comprises: a second pouch part which is filled with hydrophilic powder, and forms a negative pressure by performing a gelation reaction in combination of the hydrophilic powder with the liquid; and a first valve part connecting the first pouch part with the second pouch part.

3. The biomolecule detection device of claim 2, wherein the pressing region transmits the negative pressure formed by the gelation reaction to a flow path connected to the first pouch part.

4. The biomolecule detection device of claim 2, wherein the moving region comprises: a first valve part connected to the first pouch part; an impeller part connected to the first valve part and including a plurality of impellers; and a second valve part connecting the impeller part with the sensing region.

5. The biomolecule detection device of claim 4, wherein the impeller part comprises connection parts provided between the plurality of impellers and connecting different impellers with each other.

6. The biomolecule detection device of claim 5, wherein each of the impellers comprises: an impeller body connected to the corresponding connection part and through which the liquid passes through a circular space; an impeller center part; and a plurality of impeller blades arranged in a circumferential direction from the impeller center part.

7. The biomolecule detection device of claim 6, wherein the liquid is introduced into the impeller body through the corresponding connection part, and the backflow of the liquid is prevented while rotating in one direction by the impeller blades.

8. The biomolecule detection device of claim 2, wherein the blood collection region comprises: a needle part including a hollow needle to collect blood; a blood collection storage part in which the collected blood is stored; a separation part including a filter to separate plasma from the blood; a lamination part stacking blood from which the plasma is separated; and a channel part provided on one side of the separation part and configured in a zigzag shape to carry the plasma.

9. The biomolecule detection device of claim 8, wherein the blood is moved in the blood collection region in the direction of the negative pressure.

10. The biomolecule detection device of claim 8, wherein the sensing region comprising: a sensing part having a concave groove so that the plasma is filled, and a sensor unit in which a plurality of electrodes are deposited on the sensor unit to sense biomolecules of the plasma.

11. The biomolecule detection device of claim 8, wherein the negative pressure moves the blood inhaled in the blood collection region to the sensing region with a force in which the liquid positioned in the moving region flows backward in a direction toward the first pouch part.

12. The biomolecule detection device of claim 1, wherein the substrate of the stretchable material comprises an upper layer and a lower layer bonded to each other by a solvent bond.
